(19) United States
(12) Patent Application Publication
(10) Pub. No.: US 2011/0117182 A1
(43) Pub. Date: May 19, 2011

(54) COMBINATION OF DAPSONE WITH OTHER ANTI-ACNE AGENTS

(75) Inventors: Gurpreet Ahluwalia, Irvine, CA (US); Kevin S. Warner, Anaheim, CA (US); Haigang Chen, Petaluma, CA (US); Meidong Yang, Richmond, CA (US)

(73) Assignee: ALLERGAN, INC., Irvine, CA (US)

(21) Appl. No.: 12/846,079

(22) Filed: Jul. 29, 2010

Related U.S. Application Data
(60) Provisional application No. 61/229,903, filed on Jul. 30, 2009.

(51) Int. Cl. A61K 31/192; A61K 9/127; A61P 17/00; A61P 17/10; A61P 17/02

(52) U.S. Cl. 424/450; 514/569

(57) ABSTRACT

A composition suitable for topical application that contains at least two active ingredients, one of these being dapsone and one selected from the group consisting of adapalene, tazarotene and treinion for the effective treatment of acne and other dermatological conditions.

### CROSS REFERENCE

This application claims the benefit of U.S. Provisional Patent Application Ser. No. 61/229,903 filed on Jul. 30, 2009, the entire disclosure of which is incorporated herein by this specific reference.

### FIELD OF THE INVENTION

The present invention is directed to compositions and methods for the treatment of acne vulgaris and other dermatological conditions.

### BACKGROUND OF THE INVENTION

Acne is the most common skin disease that affects a large number of adolescents and young adults after they reach puberty. Though not a life threatening disease, it has serious psychological impact on the patient. Chronic inflammatory acne can also result in permanent scarring of the face.

There are multiple factors that contribute to the pathogenesis of acne, these include: 1. over activity of sebum production as a result of hormonal changes at puberty; 2. colonization of Propionibacterium acnes (P. acnes) in the pilosebaceous unit; 3. hyperkeratinization or abnormal desquamation of epithelium of the upper follicle (above the sebaceous gland) that results in blockage of the pilosebaceous canal; 4. formation of inflammatory molecules as a result of the action of P. acnes on sebaceous lipids.

The obstruction of the pilosebaceous canal and inflammation caused by P. acnes created inflammatory metabolites results in the formation of comedones. Excess sebum production as a result of hormonal changes at puberty, combined with increased epithelium turnover of the upper follicle leads to formation of microcomedones which progresses to inflammatory papules and pustules in acne. The combination of lipid rich sebum and protein rich desquamated cells provides an ideal environment for the growth and activity of P. acnes which converts the sebaceous lipids to the inflammatory free fatty acid molecules resulting in inflammatory acne lesions. The patient can have either non-inflammatory (open and closed comedones), inflammatory (papules and pustules) or a combination of both which most often is the case. Topical treatments are generally sufficient in most patients to control the acne lesions.

Because acne is a multifactorial condition, the marketed products work on one or more of the underlying factors contributing to acne for its treatment. There are number of prescription and over-the-counter (OTC) products available that treat acne; however, they all lack either desired efficacy or tolerability or both. Currently available products include antibiotics (topical and systemic), benzoyl peroxide, retinoids (topical and systemic), dapsone, and a number of other compounds.

The anti-acne molecule dapsone is marketed as a commercial product Aczone®. Aczone® is a 5% dapsone gel with a gritty texture due to insoluble particles of dapsone drugs. The insolubility of dapsone limits the bioavilability of dapsone upon application and its absorption through the skin and is therefore administered twice daily. At the biochemical and molecular level, dapsone exhibits an anti-inflammatory activity which provides a unique mechanism of action for this molecule in treatment of inflammatory acne lesions. However, its mechanism of action is not entirely understood. A complex combination of inflammatory pathways produce the clinical inflammation observed in acne. It is known that neutrophils significantly contribute to inflammatory acne. Dapsone is known to suppress neutrophil recruitment & local production of toxic products there by inhibiting neutrophil chemotaxis and reducing generation of oxygen free radicals. It further inhibits release of lysosomal enzymes and reduces release and bocks inflammatory effects of prostaglandins & leukotrienes. These effects results in reduction of inflammatory acne lesions. In addition to its anti-inflammatory activity, dapsone is also effective against P. acnes. MIC90 against P. acnes is 8 μg/ml.

Adapalene is a third generation retinoid, which are compounds related to Vitamin A, and has been approved by the FDA for the treatment of acne. Adapalene is known to moderate inflammatory processes but its mechanism of action is also not entirely understood. Adapalene products are sold with the concentrations of 0.1% and 0.3% w/v concentrations for gels and 0.1% w/v concentration for cream. Adapalene acts on retinoid receptors and appears to be a modifier of cellular differentiation, keratinization and inflammatory processes which are involved in the pathology of acne vulgaris. Absorption of adapalene from either 0.1% or 0.3% gel or cream is low. In one pharmacokinetic study, 16 patients suffering from acne vulgaris received 0.3% adapalene gel applied to the face, chest and back which is approximately a dosage of 2 mg/cm2. Fifteen patients resulted in quantifiable (LOQ=0.1 ng/mL) adapalene levels with a mean Cₘₐₓ of 0.553±0.466 ng/mL on Day 10 of treatment. Mean AUC₀₋₂₄ hr was 8.37±8.46 ng.h/mL as determined in 15 of the 16 patients on Day 10. Terminal apparent half-life, which was determined in 15 of 16 patients, ranged from 7 to 51 hours, with a mean of 17.2±10.2 hours. Adapalene was rapidly cleared from plasma and was not detected 72 hours after the last application for all but one subject.

### SUMMARY OF THE INVENTION

There is an unmet consumer need for an efficacious product for the treatment of acne vulgaris as the currently available products for treatment of acne vulgaris lack the desired efficacy and/or have side effects or tolerability issues that are undesired by the subjects.

A combination acne product would provide the benefit of enhanced efficacy compared to the products containing single active agent by taking advantage of the synergistic mechanism of action of the active agents for treatment of acne. The present invention is directed to acne products with at least two active compounds and in particular are directed to dapsone and adapalene combination formulations for the use in the treatment of dermatological conditions such as acne vulgaris, rosacea, atopic dermatitis, treatment of chronic wounds, bed sores, keratosis piralis, psoriasis, cosmetic improvement of surgical and acne scars, sebaceous cysts, inflammatory dermatoses, post inflammatory hyperpigmentation, eczema, xerosis, pruritis, lichen planus, nodular prurigo, eczema, and miliaria and other dermatological conditions.

Some embodiments of the present invention include:

1) A dermatological composition comprising dapsone, adapalene, and water.

2) The dermatological composition of paragraph 1 wherein the composition comprises 5% w/w dapsone and 0.1% or 0.3% w/w adapalene and is used for the treatment of acne vulgaris.

3) The dermatological composition of paragraph 2 wherein the composition is 0.5% w/w dapsone and 0.3% w/w adapalene.

4) The dermatological composition of paragraph 1 wherein the composition is a gel.

5) The compositions of paragraphs 1 and 4 wherein the composition is 0.5% w/w dapsone, 0.1% w/w adapalene, 1.5% w/w benzyl alcohol, transcutol, 5-25% w/w PEG 400, 0.01% w/w EDTA, and 0.03% w/w citric acid.

6) The compositions of paragraphs 1-5 wherein the composition further comprises hydroxyl ethyl cellulose 1-4% w/w.

7) The compositions of paragraphs 1-5 further comprising carbopol 980 at 0.5-2% w/w.

8) The compositions of paragraphs 1-7 further comprising methyl paraben.

9) The compositions of paragraphs 1-8 further comprising lactic acid.

10) The compositions of paragraphs 1-9 further comprising glycerin.

11) The composition of paragraph 5 further comprising dimethyl isosorbide in 5-15% w/w.

12) The composition of paragraphs 1-5 wherein transcutol is present in the amount of 25% w/w.

13) The compositions of paragraphs 1-12 wherein a buffer selected from the group consisting of NaOH, trolamine, and hycrochloric acid is added to adjust the pH.

14) The compositions of paragraphs 1-13 wherein the pH of the composition is 5.5.

15) The composition of paragraphs 1-5 further comprising 2-3% hydroxyl ethyl cellulose.

16) The compositions of paragraphs 1-15 wherein the composition is in the form of one selected from the group consisting of a gel, emulsion, cream, liquid, paste, lotion, nanoemulsion, microemulsion, reverse emulsion and liposomal cream.

17) The compositions of paragraphs 1-16 wherein the composition may be used for treatment of one selected from the group consisting of acne vulgaris, rosacea, atopic dermatitis, treatment of chronic wounds, bed sores, keratosis piralis, sebaceous cysts, inflammatory dermatoses, post inflammatory hyperpigmentation, eczema, xerosis, pruritis, lichen planus, nodular prurigo, dermatitis, eczema, and miliaria and other dermatological conditions.

18) A method of treating acne vulgarus by application of the compositions of paragraphs 1-17.

19) The method of treatment of paragraph 17, wherein the application is once a day.

20) The method of treatment of paragraph 17, wherein the application is twice a day.

### BRIEF DESCRIPTION OF THE DRAWINGS

FIG. 1 is directed to dapsone and adapalene formulations for the treatment of dermatological conditions;

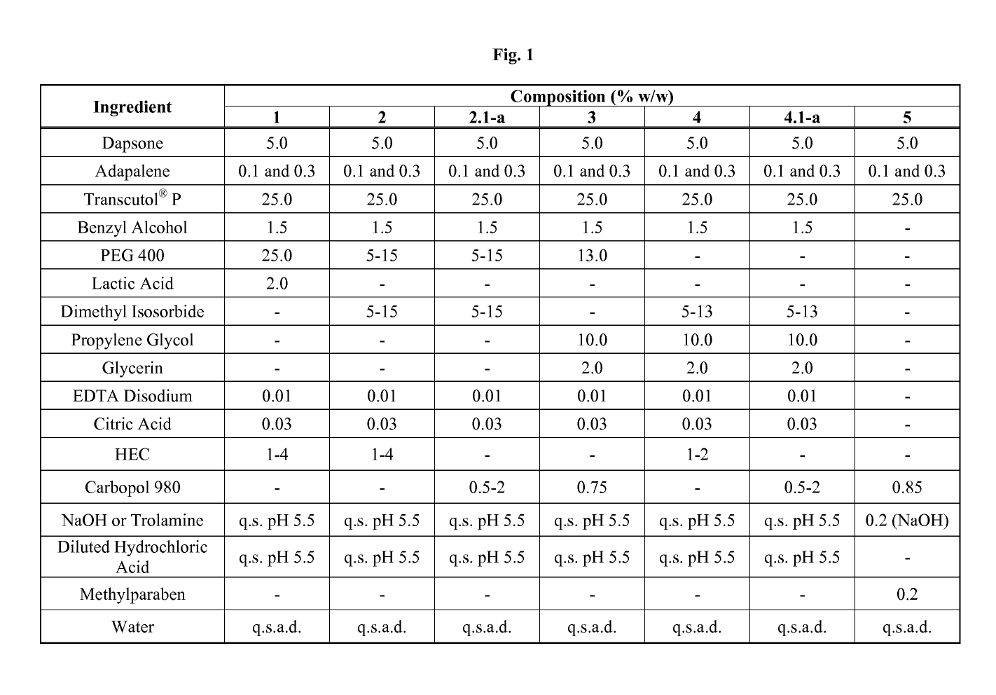

FIG. 2 is directed to variations of formulations for the treatment of dermatological conditions of Formula 1 of FIG. 1;

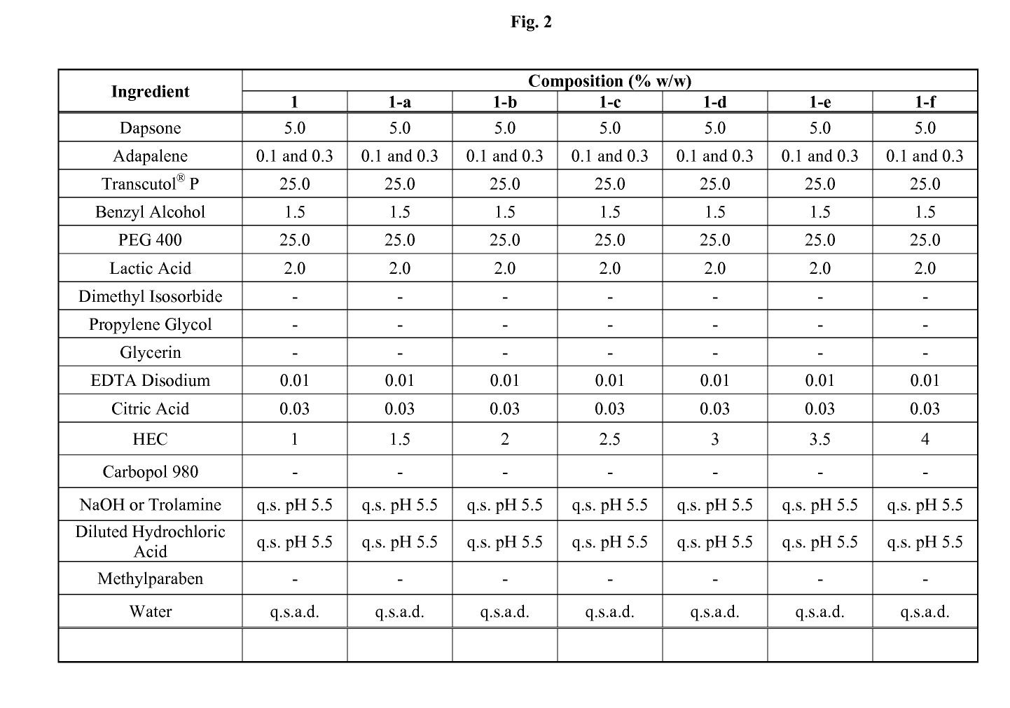

FIG. 3A is directed to variations of formulations for the treatment of dermatological conditions of Formula 2 of FIG. 1;

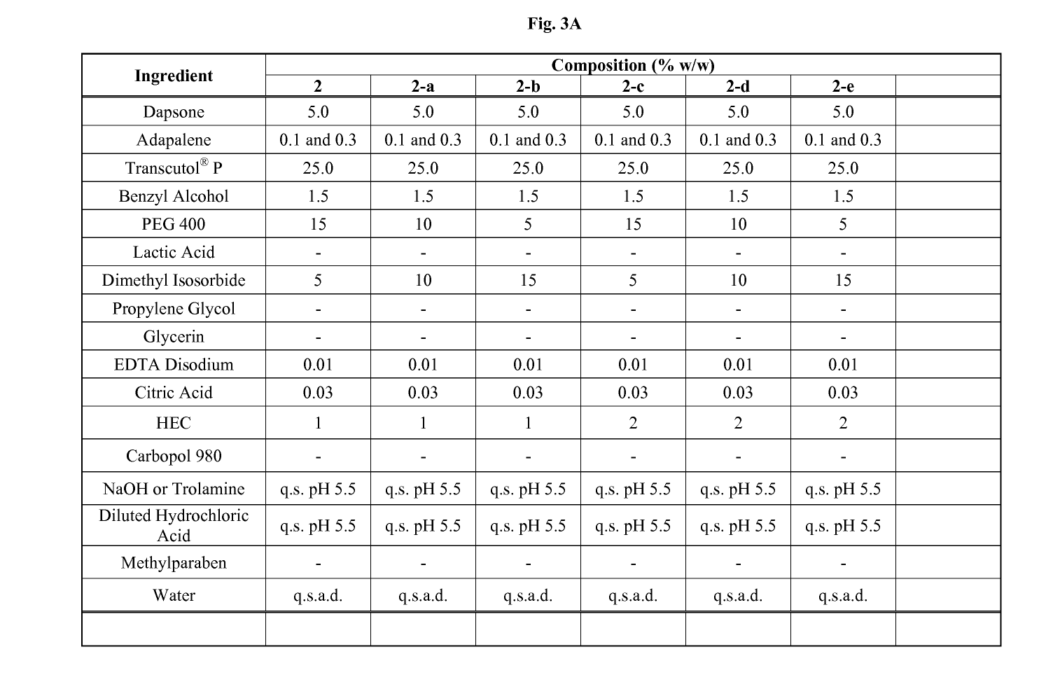

FIG. 3B is directed to variations of formulations for the treatment of dermatological conditions of Formula 2 of FIG. 1;

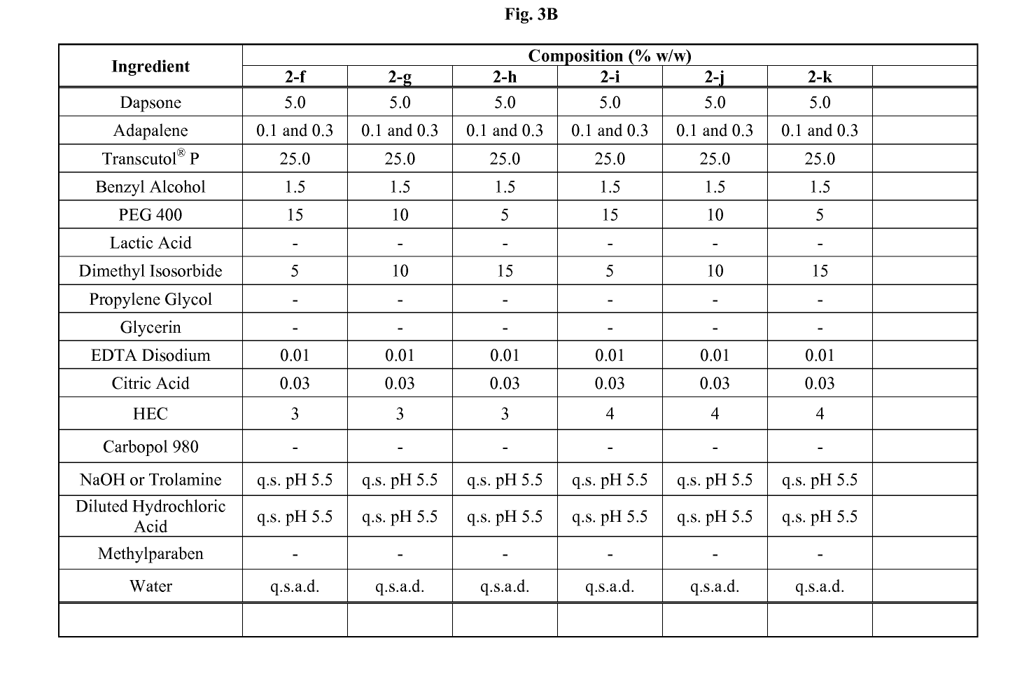

FIG. 3C is directed to variations of formulations for the treatment of dermatological conditions of Formula 2.1 of FIG. 1;

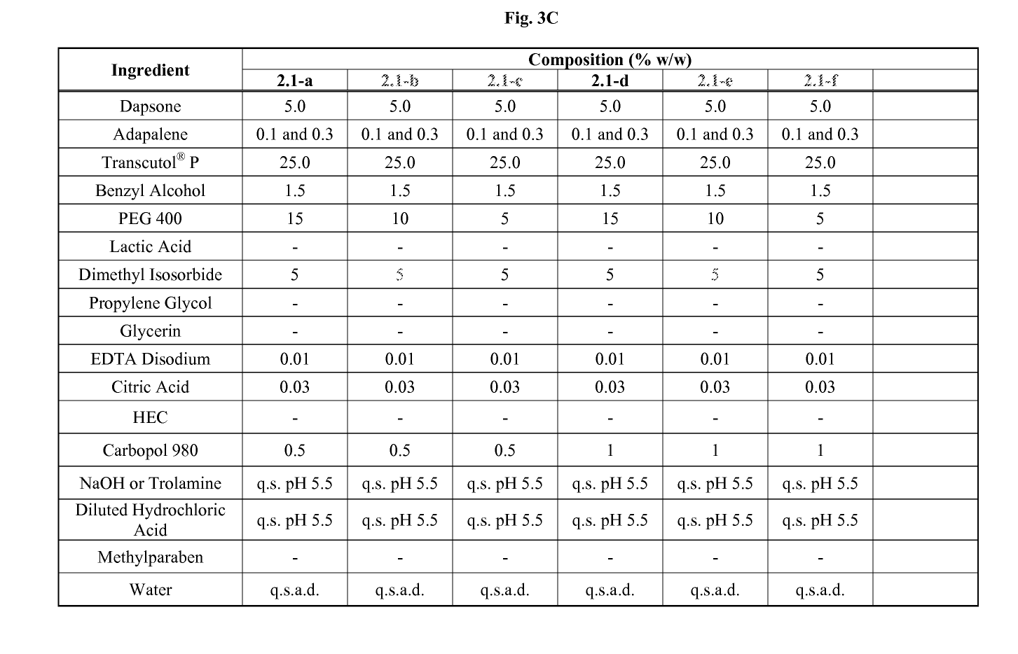

FIG. 3D is directed to variations of formulations for the treatment of dermatological conditions of Formula 2.1 of FIG. 1;

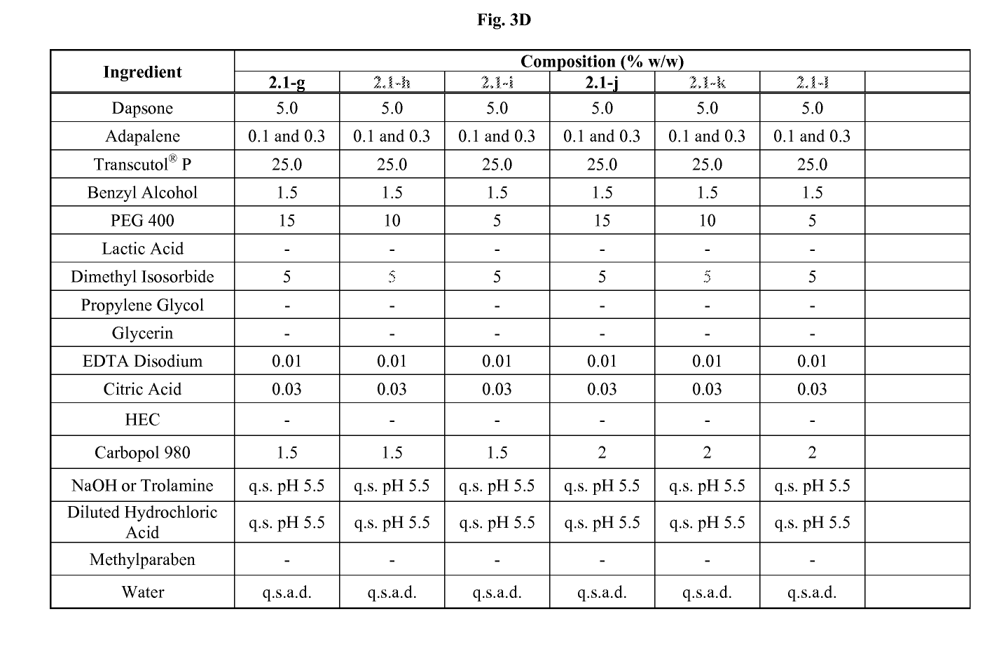

FIG. 4A is directed to variations of formulations for the treatment of dermatological conditions of Formula 4 of FIG. 1;

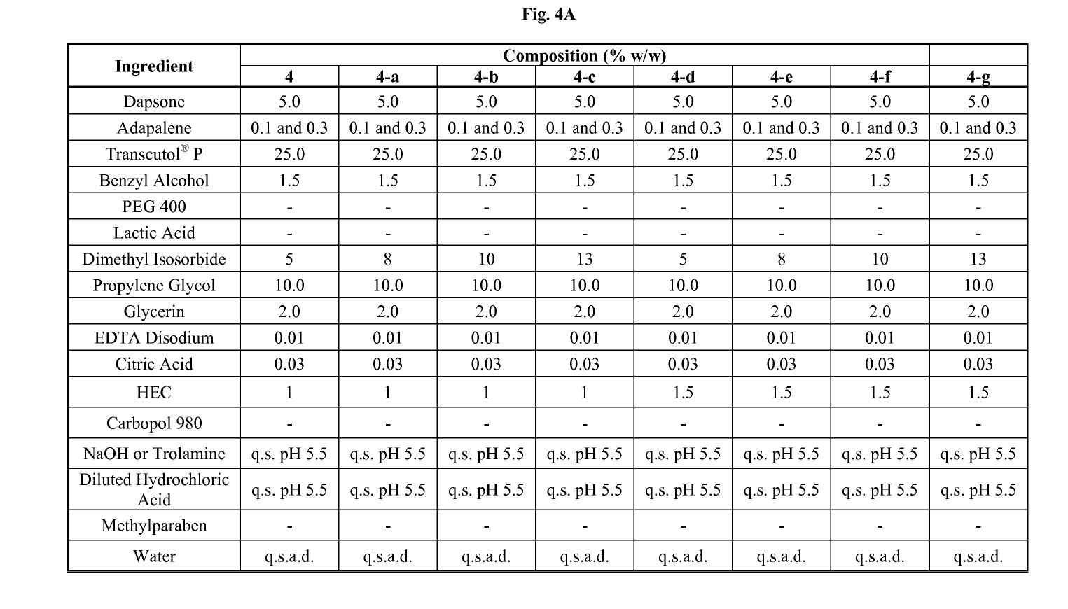

FIG. 4B is directed to variations of formulations for the treatment of dermatological conditions of Formula 4 of FIG. 1;

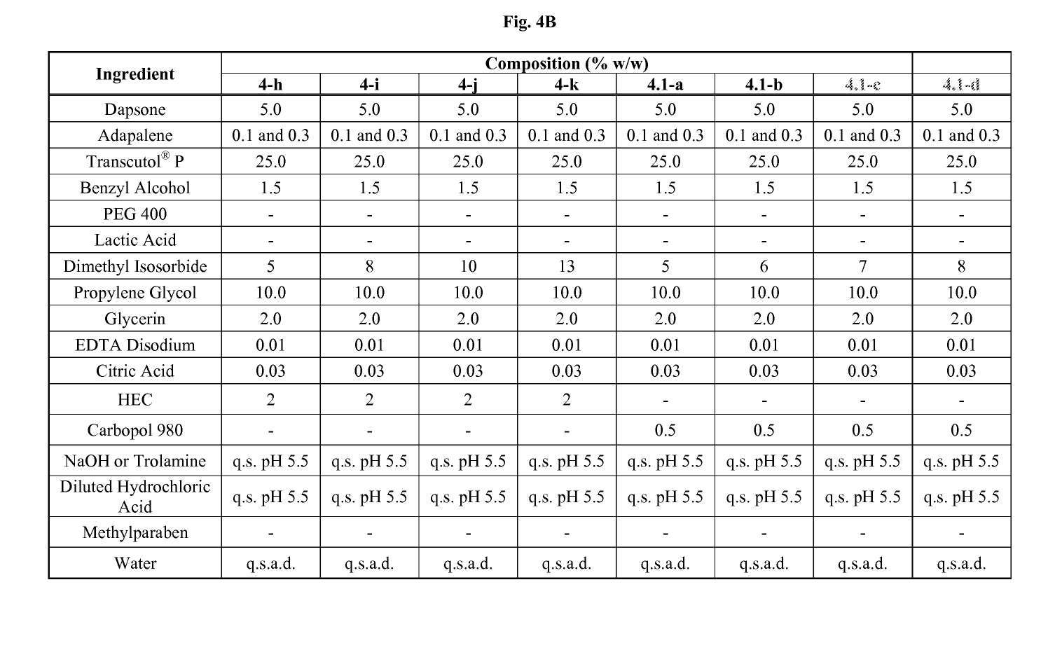

FIG. 4C is directed to variations of formulations for the treatment of dermatological conditions of Formula 4 of FIG. 1;

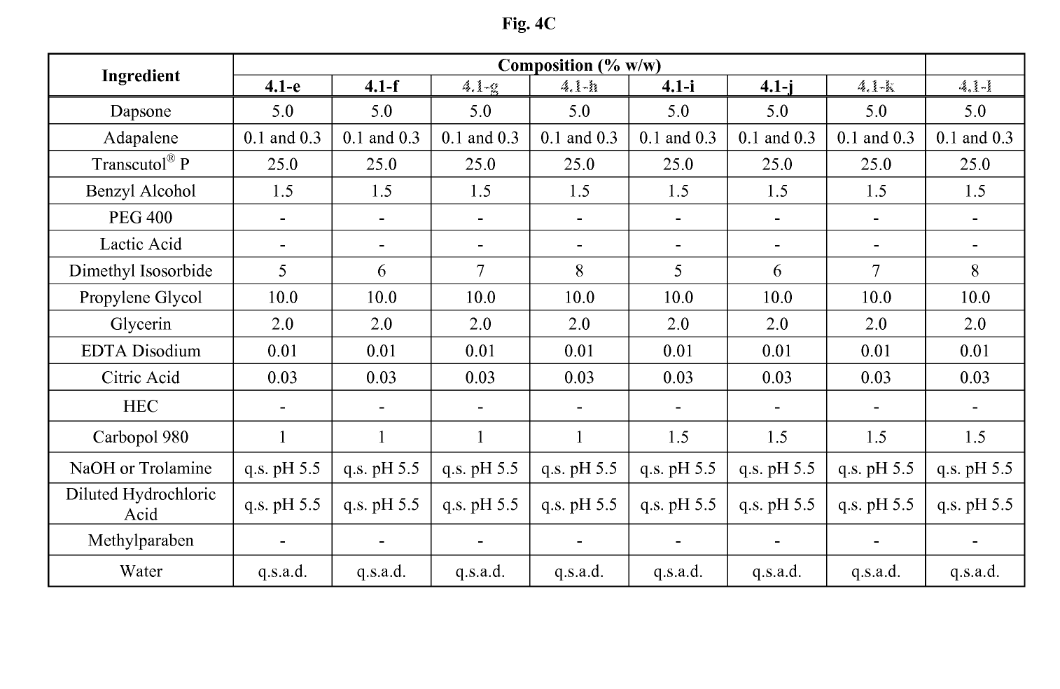

FIG. 4D is directed to variations of formulations for the treatment of dermatological conditions of Formula 4 of FIG. 1; and,

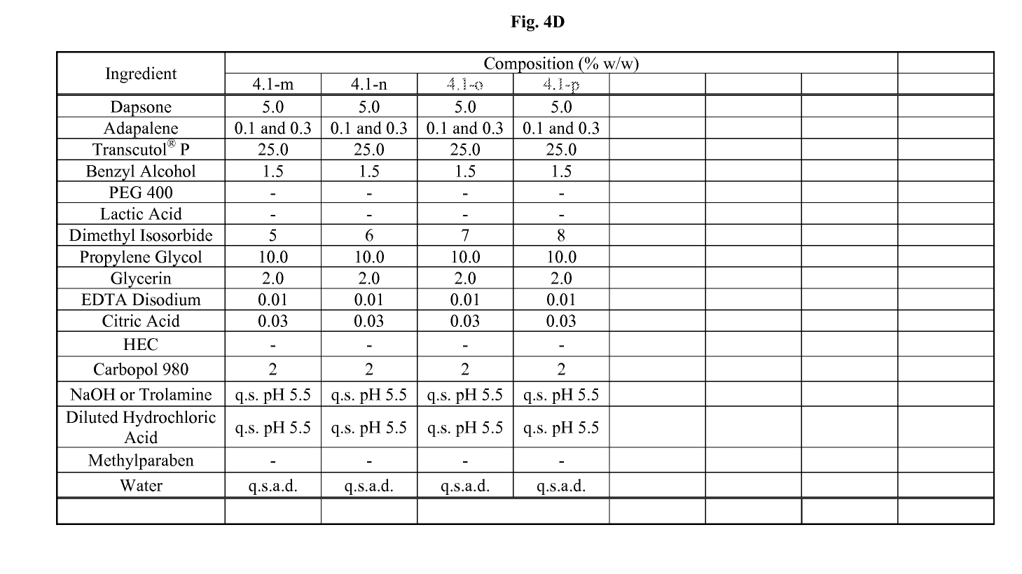

FIG. 5 is directed to dapsone and adapalene formulations for the treatment of dermatological conditions.

### DETAILED DESCRIPTION OF THE INVENTION

The present invention is directed to topical compositions for treatment of dermatological conditions which contain at least two active ingredients, one of these being dapsone and the other(s) selected from the list below for an effective treatment of acne and other dermatological conditions such as rosacea.

Some broad embodiments of the invention and possible combinations are found below:

Suitable compounds that can be combined with dapsone (2-10% w/w) include:
1. Agents with bactericidal and/or comedolytic properties:
   a. Benzoyl peroxide (2.5-10% w/w); and,
   b. other antimicrobial actives that are effective against P. acnes .
2. Agents that inhibit comedogenesis by reducing pilosebaceous canal obstruction or have keratolytic properties such as:
   a. Salicylic acid (0.5-3% w/w);
   b. Azelaic acid (up to 20% w/w);
   c. Sulfacetamide-sulfur (5-10% w/w); and,
   d. other keratolytic agents.
3. Agents that reduce sebaceous gland secretion and effect epithelial dysquamation:
   a. Retinoids:
      i. tretinoin or trans retinoic acid (0.02-0.1% w/w);
      ii. Tazarotene (0.05-0.1% w/w);
      iii. Adapalene (0.1-0.3% w/w); and,
      iv. additional retinoids.
4. Topical antibiotics for directly killing P. acnes:
   a. erythromycin (1-3% w/w);
   b. clindamycin (1-2% w/w); and,
   c. tetracycline (1-3% w/w).
      Potential combinations that can be used:

1. Dapsone (0.01%-10% w/w)+retinoid (0.001%-3% w/w)
Examples:
   a. Dapsone 5% w/w+Adapalene 0.3% w/w;
   b. Dapsone 5% w/w+tazarotene 0.1% w/w; and,
   c. Dapsone 5% w/w+tretinoin 0.1% w/w.

2. Dapsone+benzoyl peroxide:
Examples
   a. Dapsone 5% w/w+benzoyl peroxide 5% w/w;

3. Dapsone+antibiotic:
Examples
   a. Dapsone 5% w/w+clindamycin 1% w/w.

4. Dapsone+keratolytic agent
Examples:
   a. Dapsone 5% w/w+Azelaic acid 20% w/w.

The concentration values (w/w) in parenthesis represent preferred concentration; however, other concentrations values (w/v) can be used dependent on the formulation characteristics and the desired level of efficacy and tolerability.

In a recent clinical trial the safety and efficacy of dapsone gel co-administered with adapalene gel was assessed. The study design consisted of having patients apply the product Aczone® (5% w/w dapsone) twice a day, with morning and evening application. About 10 minutes after the evening application of Aczone®, patients applied a thin layer of 0.1% w/w adapalene gel. The 10 minute separation between applications of the two products ensured complete absorption of the Aczone® formulation into the skin to minimize the potential negative impact on adapalene or dapsone skin penetration. Application of the 0.1% w/w adapalene gel immediately after the Aczone® application may have resulted in a situation where the adapalene or dapsone would have a lower skin penetration because of the mixing of the two formulation vehicles. Further, the additional thickness of the combined formulation applications may increase the penetration distance of the two actives also resulting in reduced skin penetration of the actives.

The results of the trial showed that dapsone gel administered concurrently (but not together) with adapalene gel is safe and well tolerated for the treatment of acne vulgaris. One aspect of the present invention is a combination adapalene/dapsone topical formulation combining the two actives into one formulation. The novelty of this invention is in part attributable to the use of additional excipients (solubilizers) in combination with diethylene glycol monoethyl ether (“DGME”) in order to solubilize dapsone. Addition of cosolvents has enabled the complete dissolution of dapsone in the formulation and an increase in the solubility of adapalene (adapalene is not completely solubilized in these formulations). The increased concentration of dissolved dapsone and adapalene versus the marketed product comparators administered concurrently will increase the rate of skin penetration of both drugs into and through the skin

Topical dosage forms of the present invention include, but are not limited to solutions, gels, creams, ointments, foams, emulsions, films, and facial/skin peels. The present invention is directed to topical dapsone and adapalene formulations which are formulated to optimize the dermal delivery profile of adapalene and dapsone to effectively treat acne and other dermatological conditions and improve the efficiency of pharmaceutical products applied to the skin.

Examples of some formulations encompassed by the present invention excipients and concentration ranges are summarized in Table 1 below:

TABLE 1
Example Excipient Composition Ranges Utilized in Adapalene/Dapsone
Topical Formulations:

| Ingredient | Function | Composition (% w/w) |
|---|---|---|
| Dapsone | Active | 0.5-10 |
| Adapalene | Active | 0.1-0.3 |
| Carbomer 980 | Thickener | 0.05-01.5 |
| Hydroxyethyl cellulose | | 1-8% |
| Hydroxypropyl cellulose | | 1-6% |
| NaOH | Neutralizing Agent | 0.01-2.0 |
| Trolamine | Neutralizing Agent | 0.01-2.0 |
| Ethanol | Solubilizers | 1-90 |
| Lactic acid | | 1-10 |
| diethylene glycol monoethyl | | 1-50 |
| ether | | |
| propylene glycol | | 1-60 |
| Dimethyl isosorbide | | 1-30 |
| Polyethylene glycol 400 | | 1-50 |
| Hexylene glycol | | 1-50 |
| Isostearyl alcohol | | 0.5-10 |
| Medium chain triglycerides | | 0.5-10 |
| Isopropyl myristate | | 2-10 |
| Benzyl alcohol | Preservative | 0.5-5 |
| Methyl Paraben | Preservative | 0.1-0.3 |
| Propyl Paraben | Preservative | 0.01-1 |
| Benzalkonium Chloride | Preservative | 0.1-0.2 |
| Sorbic Acid | Preservative | 0.1-2.7 |
| Glycerol | Humectant | 1-20 |
| Polyvinyl alcohol | Film forming | 1-30 |
| Water | Vehicle | 1-90 |
| EDTA Disodium | Antioxidant | 0.005-0.02 |
| Citric Acid | Antioxidant | 0.015-0.06 |
| Butylated hydroxytoluene | Antioxidant | 0.005-1 |
| Butylated hydroxyanisole | Antioxidant | 0.01-0.25 |
| Propyl gallate | Antioxidant | 0.01-0.1 |
| Elastomer 10 | Thickener | 0.1-90 |
| ST Wax 30 | Thickener | 0.1-50 |
| Dimethiconol blend 20 | Thickener | 0.1-50 |
| Emulsifier 10 | Emulsifier | 0.1-50 |
| cyclomethicone 5 | Solvent | 0.1-50 |
| Silicone fluid | Solvent | 0.1-50 |
| Silky wax 10 | Thickener | 0.1-50 |

Further specific compositions of the present invention of 5% w/w dapsone and 0.1% w/w and 0.3% w/w adapalene formulations include but are not limited to:

TABLE 2A
Adapalene/Dapsone Topical Formulations

| Ingredient | Function | Composition (% w/w) | | | | | | | | |
|---|---|---|---|---|---|---|---|---|---|---|
| Dapsone | Active | 5 | 5 | 5 | 5 | 5 | 5 | 5 | 5 | 5 |
| Adapalene | Active | 0.1% | 0.1% | 0.1% | 0.1% | 0.1% | 0.1% | 0.1% | 0.1% | 0.1% |
| | | or | or | or | or | or | or | or | or | or |
| | | 0.3% | 0.3% | 0.3% | 0.3% | 0.3% | 0.3% | 0.3% | 0.3% | 0.3% |
| diethylene | Solubilizing | 25 | 20 | 25 | 20 | 25 | 25 | 25 | 25 | 25 |
| glycol | Agent | | | | | | | | | |
| monoethyl | | | | | | | | | | |
| ether | | | | | | | | | | |
| Benzyl | Preservative | 1.5 | 1.5 | 1.5 | 1.5 | 1.5 | 1.5 | 1.5 | 1.5 | — |
| Alcohol | | | | | | | | | | |
| PEG 400 | Solubilizing | 25 | 20 | 25 | 20 | 15 | — | — | — | — |
| | Agent | | | | | | | | | |
| Lactic Acid | Solubilizing | 5 | 4 | — | — | — | — | — | — | — |
| | Agent | | | | | | | | | |
| Dimethyl | Solubilizing | — | — | — | 15 | — | — | — | — | — |
| Isosorbide | Agent | | | | | | | | | |
| Propylene | Solubilizing | — | — | — | — | — | 20 | 20 | 10 | — |
| Glycol | Agent | | | | | | | | | |
| Glycerin | Humectant | — | — | — | — | — | 10 | 10 | 2 | — |
| Isopropyl | Solubilizing | — | — | — | — | — | — | — | — | 5 |
| Myristate | Agent | | | | | | | | | |
| EDTA | Antioxidant | 0.01 | 0.01 | 0.01 | 0.01 | 0.01 | 0.01 | 0.01 | 0.01 | — |
| Disodium | | | | | | | | | | |
| Citric Acid | Antioxidant | 0.03 | 0.03 | 0.03 | 0.03 | 0.03 | 0.03 | 0.03 | 0.03 | — |
| Hydroxyethyl | Thickener | 4 | 3 | | — | 4 | — | — | — | — |
| Cellulose | | | | | | | | | | |
| Carbopol 980 | Thickener | — | — | — | 0.75 | — | 0.75 | 0.75 | 0.75 | — |
| Hydroxypropyl | Thickener | — | — | — | — | — | — | — | — | 3 |
| Cellulose | | | | | | | | | | |
| NaOH | Neutralizing | 1.5 | 1.2 | q.s. | q.s. | q.s. | q.s. | q.s. | q.s. | — |
| | Agent | | | pH 5.5 | pH 5.5 | pH 5.5 | pH 5.5 | pH 5.5 | pH 5.5 | |
| Diluted | Neutralizing | — | — | q.s. | q.s. | q.s. | q.s. | q.s. | q.s. | — |
| Hydrochloric | Agent | | | pH 5.5 | pH 5.5 | pH 5.5 | pH 5.5 | pH 5.5 | pH 5.5 | |
| Acid | | | | | | | | | | |
| Ethanol | Solubilizer | — | — | — | — | — | — | — | — | 60 |
| Water | Vehicle | q.s.a.d. | q.s.a.d. | q.s.a.d. | q.s.a.d. | q.s.a.d. | q.s.a.d. | q.s.a.d. | q.s.a.d. | — |

TABLE 2B
Adapalene/Dapsone Topical Formulations (cont.)

| Ingredient | Function | Composition (% w/w) | | |
|---|---|---|---|---|
| Dapsone | Active | 5 | 5 | 5 |
| Adapalene | Active | 0.1% | 0.1% | 0.1% |
| | | or | or | or |
| | | 0.3% | 0.3% | 0.3% |
| diethylene | Solubilizing | 25 | 25 | 25 |
| glycol | Agent | | | |
| monoethyl | | | | |
| ether | | | | |
| Benzyl Alcohol | Preservative | 1.5 | 1.5 | 1.5 |
| PEG 400 | Solubilizing | 13 | — | — |
| | Agent | | | |
| Dimethyl | Solubilizing | — | 13 | 13 |
| Isosorbide | Agent | | | |
| Propylene Glycol | Solubilizing | 15 | 15 | 15 |
| | Agent | | | |
| Glycerin | Humectant | 2 | 2 | 2 |
| EDTA Disodium | Antioxidant | 0.01 | 0.01 | 0.01 |
| Citric Acid | Antioxidant | 0.03 | 0.03 | 0.03 |
| Hydroxyethyl | Thickener | — | 2 | — |
| Cellulose | | | | |
| Carbopol 980 | Thickener | 0.75 | — | — |
| Hydroxypropyl | Thickener | — | — | 2 |
| Cellulose | | | | |
| NaOH | Neutralizing | q.s. | q.s. | q.s. |
| | Agent | pH 5.5 | pH 5.5 | pH 5.5 |
| Diluted | Neutralizing | q.s. | q.s. | q.s. |
| Hydrochloric | Agent | pH 5.5 | pH 5.5 | pH 5.5 |
| Acid | | | | |
| Water | Vehicle | q.s.a.d. | q.s.a.d. | q.s.a.d. |

The formulations of the present invention can be made as follows based on the excipients:

Process for making lactic acid containing formulations:

The combination adapalene/dapsone gels were prepared as follows:
a. Weigh the Transcutol into a kettle. Add the dapsone, lactic acid, polyethylene glycol 400, benzyl alcohol. Stir with propeller mixer at room temperature. Mix until dissolved;
b. Add water, EDTA, and citric acid to mixture in step a. Mix until dissolved;
c. Add adapalene to mixture in step b;
d. While continuing to mix, slowly add hydroxyethyl cellulose to mixture in step c avoid clumping. Mix vigorously at room temperature until a uniform lump-free dispersion is achieved; and,
e. While mixing add sufficient sodium hydroxide to achieve a pH of 5.3 to 5.7. Mix until uniform.
   Process for making DMI/hydroxyethyl cellulose containing formulations:
   The combination adapalene/dapsone gels were prepared as follows:
a. Weigh the Transcutol into a kettle. Add the dapsone, dimethyl isosorbide, polyethylene glycol 400, benzyl alcohol. Stir with propeller mixer at room temperature. Mix until dissolved;
b. Add water, EDTA, and citric acid to mixture in step a. Mix until dissolved.
c. Add adapalene to mixture in step b;
d. While continuing to mix, slowly add hydroxyethyl cellulose to mixture in step c avoid clumping. Mix vigorously at room temperature until a uniform lump-free dispersion is achieved; and,
e. While mixing add sufficient sodium hydroxide to achieve a pH of 5.3 to 5.7. Mix until uniform.
   Process for making DMI/Carbopol containing formulations:
   The combination adapalene/dapsone gels were prepared as follows:
a. Weigh the Transcutol into a kettle. Add the dapsone, dimethyl isosorbide, polyethylene glycol 400, benzyl alcohol. Stir with propeller mixer at room temperature. Mix until dissolved;
b. Add water, EDTA, and citric acid to mixture in step a. Mix until dissolved;
c. Add adapalene to mixture in step b;
d. While continuing to mix, slowly add Carbopol 980 to mixture in step c avoid clumping. Mix vigorously at room temperature until a uniform lump-free dispersion is achieved; and,
e. While mixing add sufficient sodium hydroxide to achieve a pH of 5.3 to 5.7. Mix until uniform.
   Process for making PG/PEG containing formulations:
   The combination adapalene/dapsone gels were prepared as follows:
a. Weigh the Transcutol into a kettle. Add the dapsone, propylene glycol, polyethylene glycol 400, benzyl alcohol. Stir with propeller mixer at room temperature. Mix until dissolved;
b. Add water, EDTA, and citric acid to mixture in step a. Mix until dissolved;
c. Add adapalene to mixture in step b;
d. While continuing to mix, slowly add Carbopol 980 to mixture in step c avoid clumping. Mix vigorously at room temperature until a uniform lump-free dispersion is achieved; and,
e. While mixing add sufficient sodium hydroxide to achieve a pH of 5.3 to 5.7. Mix until uniform.
   Process for making PG/DMI/Carbopol containing formulations:
   The combination adapalene/dapsone gels were prepared as follows:
a. Weigh the Transcutol into a kettle. Add the dapsone, propylene glycol, dimethyl isosorbide, benzyl alcohol. Stir with propeller mixer at room temperature. Mix until dissolved;
b. Add water, EDTA, and citric acid to mixture in step a. Mix until dissolved;
c. Add adapalene to mixture in step b;
d. While continuing to mix, slowly add Carbopol 980 to mixture in step c avoid clumping. Mix vigorously at room temperature until a uniform lump-free dispersion is achieved; and,
e. While mixing add sufficient sodium hydroxide to achieve a pH of 5.3 to 5.7. Mix until uniform.
   Process for making PG/DMI/HEC containing formulations:
   The combination adapalene/dapsone gels were prepared as follows:
a. Weigh the Transcutol into a kettle. Add the dapsone, propylene glycol, dimethyl isosorbide, benzyl alcohol. Stir with propeller mixer at room temperature. Mix until dissolved;
b. Add water, EDTA, and citric acid to mixture in step a. Mix until dissolved;
c. Add adapalene to mixture in step b;
d. While continuing to mix, slowly add hydroxyethyl cellulose to mixture in step c avoid clumping. Mix vigorously at room temperature until a uniform lump-free dispersion is achieved; and,
e. While mixing add sufficient sodium hydroxide to achieve a pH of 5.3 to 5.7. Mix until uniform.

The most effective dapsone and adapalene composition is selected based on clinical studies. For example, a clinical study is conducted by forming two treatment groups, one with daily application of a selected dapsone and adapalene formulation, and twice daily topical application of the same selected dapsone and adapalene formulation to the acne area of the skin for a period of 12 weeks. Two control groups are formed with application once and twice daily of a vehicle consisting of the same excipients but no active ingredients. The patient's inflammatory and non-inflammatory acne lesion counts should be recorded at baseline before initiation of treatment and then at select intervals throughout the study. The reduction in total, non-inflammatory or inflammatory lesions counts provides determination of the efficacy of the formulations. The established Global Acne Assessment Score (GAAS) should be used to assess efficacy of the product. The tolerability of the product can be determined by assessment of skin dryness, irritation, sensitivity and redness as a result of treatment. A product is considered to have better tolerability if there is less effect on these parameters.

### Application Method:

1. A suitable application method is topical cream, gel, lotion, ointment, foam, liquid or a semi solid preparation. A topical preparation may contain additional ingredients to provide aesthetic and moisturizing and anti-inflammatory benefits to the skin. Generally,
   a. A gel or liquid preparation can be alcohol or aqueous based or a combination of two;
   b. A nanoemulsion or microemulsion preparation can be used for enhanced delivery of actives;
   c. A liposomal cream or lotion preparation can be used for enhanced delivery of actives; and
   d. A foam preparation can be a quick breaking foam with additional emollient components.
2. Topical preparations that result in slow release or controlled release of the active agent can also be used to provide an optimal efficacy and tolerability balance.
3. Active ingredients encapsulated in micro beads or adsorbed on microsponge can be used for control release and in addition solve any incompatibility issues between the formulation ingredients.
4. The application is preferably once a day or more frequent depending on the desired effect.

### Application of the Formulations of the Present Invention

### Example #1

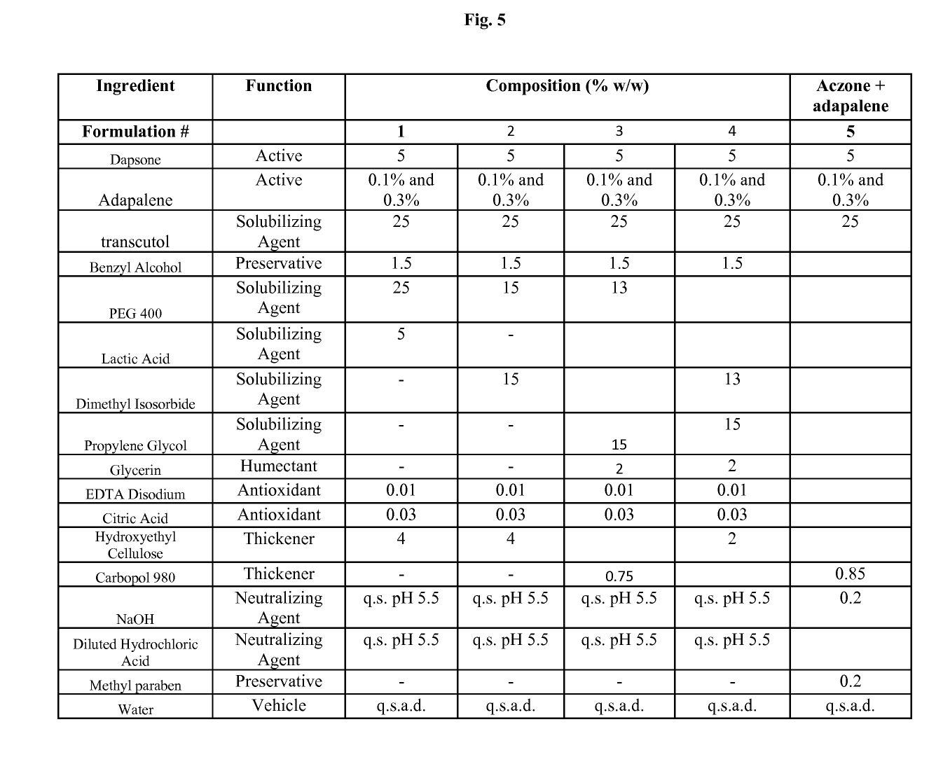

### Application of 0.1% w/w Adapalene of Formula 1 in FIG. 5

A 17 year old Caucasian male patient suffers acne vulgaris with a combination of inflammatory and non-inflammatory lesions and applies a 0.1% w/w adapalene formulation according to formulation #1 in FIG. 5. The 17 year old male patient applies the 0.1% w/w adapalene composition of Formula 1 once daily for 12 weeks. After 12 weeks, the 17 year old male patient experiences a 32% reduction in inflammatory and non-inflammatory lesions.

### Example #2

### Application of 0.3% w/w Adapalene of Formula 1 in FIG. 5

A 16 year old Caucasian female patient suffers acne vulgaris with a combination of inflammatory and non-inflammatory lesions and applies a 0.3% w/w adapalene formulation according to formulation #1 in FIG. 5. The 16 year old female patient applies the 0.3% w/w adapalene composition of Formula 1 once daily for 12 weeks. After 12 weeks, the 16 year old female patient experiences a 41% reduction in inflammatory and non-inflammatory lesions.

### Example #3

### Application of 0.1% w/w Adapalene of Formula 2 in FIG. 5

A 23 year old African-American female patient suffers acne vulgaris with a combination of inflammatory and non-inflammatory lesions and applies a 0.1% w/w adapalene formulation according to formulation #2 in FIG. 5. The 23 year old female patient applies the 0.1% w/w adapalene composition of Formula 2 once daily for 12 weeks. After 12 weeks, the 23 year old female patient experiences a 24% reduction in inflammatory and non-inflammatory lesions.

### Example #4

### Application of 0.3% w/w Adapalene of Formula 2 in FIG. 5

A 19 year old Caucasian female patient suffers acne vulgaris with a combination of inflammatory and non-inflammatory lesions and applies a 0.3% w/w adapalene formulation according to formulation #2 in FIG. 5. The 19 year old female patient applies the 0.3% w/w adapalene composition of Formula 2 once daily for 12 weeks. After 12 weeks, the patient experiences a 248% reduction in inflammatory and non-inflammatory lesions.

### Example #5

### Application of 0.1% w/w Adapalene of Formula 3 in FIG. 5

An 18 year old African-American male patient suffers acne vulgaris with a combination of inflammatory and non-inflammatory lesions and applies a 0.1% w/w adapalene formulation according to formulation #3 in FIG. 5. The 18 year old male patient applies the 0.1% w/w adapalene composition once daily for 12 weeks. After 12 weeks, the 18 year old male patient experiences a 29% reduction in inflammatory and non-inflammatory lesions.

### Example #6

### Application of 0.3% w/w Adapalene of Formula 3 in FIG. 5

An 23 year old Asian female patient suffers acne vulgaris with a combination of inflammatory and non-inflammatory lesions and applies a 0.3% w/w adapalene formulation according to formulation #3 in FIG. 5. The 23 year old patient applies the 0.3% w/w adapalene composition once daily for 12 weeks. After 12 weeks, the patient experiences a 25% reduction in inflammatory and non-inflammatory lesions.

### Example #7

### Application of 0.1% w/w Adapalene of Formula 4 in FIG. 5

An 18 year old African-American male patient suffers acne vulgaris with a combination of inflammatory and non-inflammatory lesions and applies a 0.1% w/w adapalene formulation according to formulation #3 in FIG. 5. The 18 year old male patient applies the 0.1% w/w adapalene composition once daily for 12 weeks. After 12 weeks, the 18 year old male patient experiences a 29% reduction in inflammatory and non-inflammatory lesions.

### Example #8

### Application of 0.3% w/w Adapalene of Formula 4 in FIG. 5

A 17 year old Caucasian female patient suffers acne vulgaris with a combination of inflammatory and non-inflammatory lesions and applies a 0.3% w/w adapalene formulation according to formulation #4 in FIG. 5. The 17 year old male patient applies the 0.3% w/w adapalene composition twice daily for 12 weeks. After 12 weeks, the 17 year old male patient experiences a 41% reduction in inflammatory and non-inflammatory lesions.

### Example #9

### Application of 0.1% w/w Adapalene of Formula 5 in FIG. 5

A 16 year old Caucasian female patient suffers acne vulgaris with a combination of inflammatory and non-inflammatory lesions and applies a 0.1% w/w adapalene formulation according to formulation #5 in FIG. 5. The 16 year old female patient applies the 0.1% w/w adapalene composition once daily for 12 weeks. After 12 weeks, the patient experiences a 27% reduction in inflammatory and non-inflammatory lesions.

### Example #10-Example #9

### Application of 0.3% w/w Adapalene of Formula 5 in FIG. 5

A 19 year old Caucasian female patient suffers acne vulgaris with a combination of inflammatory and non-inflammatory lesions and applies a 0.3% w/w adapalene formulation according to formulation #5 in FIG. 5. The 19 year old female patient applies the 0.3% w/w adapalene composition twice daily for 12 weeks. After 12 weeks, the patient experiences a 38% reduction in inflammatory and non-inflammatory lesions.

### Example #11

### Application of 0.1% w/w Adapalene of Formula 1 in FIG. 5

A 37 year old Caucasian male patient suffers from rosacea and applies a 0.1% w/w adapalene formulation according to formulation #1 in FIG. 5. The 37 year old male patient applies the 0.1% w/w adapalene composition of Formula 1 once daily for 12 weeks. After 12 weeks, the 37 year old male patient experiences a reduction in the symptoms of rosacea.

1) A dermatological composition comprising dapsone, adapalene, and water.

2) The dermatological composition of claim 1 wherein the 1 composition comprises 5% w/w dapsone and 0.1% w/w adapalene and is used for the treatment of acne vulgaris.

3) The dermatological composition of claim 2 wherein the composition is 0.5% w/w dapsone and 0.3% w/w adapalene.

4) The dermatological composition of claim 1 wherein the composition is a gel.

5) The composition of claim 1 wherein the composition is 0.5% w/w dapsone, 0.1% w/w adapalene, 1.5% w/w benzyl alcohol, transcutol, 5-25% w/w PEG 400, 0.01% w/w EDTA and 0.03% w/w citric acid.

6) The composition of claim 5 wherein the composition further comprises hydroxyl ethyl cellulose 1-4% w/w.

7) The composition of claim 5 further comprising carbopol 980 at 0.5-2% w/w.

8) The composition of claim 5 further comprising methyl paraben.

9) The composition of claim 5 further comprising lactic acid.

10) The composition of claim 5 further comprising glycerin.

11) The composition of claim 5 further comprising dimethyl isosorbide at 5-15% w/w.

12) The composition of claim 5 wherein transcutol is present in the amount of 25% w/w.

13) The composition of claim 5 wherein a buffer selected from the group consisting of NaOH, trolamine, and hycrochloric acid is added to adjust the pH.

14) The composition of claim 13 wherein the pH of the composition is 5.5.

15) The composition of claim 5 further comprising 2-3% hydroxyl ethyl cellulose.

16) The composition of claim 1 wherein the composition is in the form of one selected from the group consisting of a gel, emulsion, cream, liquid, paste, lotion, nanoemulsion, microemulsion, reverse emulsion and liposomal cream.

17) The composition of claim 5 wherein the composition may be used for treatment of one condition selected from the group consisting of acne vulgaris, rosacea, atopic dermatitis, treatment of chronic wounds, bed sores, keratosis piralis, sebaceous cysts, inflammatory dermatoses, post inflammatory hyperpigmentation, eczema, xerosis, pruritis, lichen planus, nodular prurigo, dermatitis, eczema, and miliaria and other dermatological conditions.

18) A method of treating acne vulgarus by application of the composition of claim 1.

19) The method of treatment of claim 17, wherein the application is once a day.

20) The method of treatment of claim 17, wherein the application is twice a day.
